(19) United States
(12) Patent Application Publication
(10) Pub. No.: US 2026/0345468 A1
(43) Pub. Date: Nov. 5, 2026

(54) FACIAL CARE FORMULATIONS WITHOUT WATER

(71) Applicant: Jean-François BERNIER, Westmount (CA)

(72) Inventor: Hans DROUIN, Tingwick (CA)

(21) Appl. No.: 19/118,875

(22) PCT Filed: Sep. 29, 2023
(86) PCT No.: PCT/CA2023/051293

§ 371 Date: Apr. 7, 2025

Related U.S. Application Data
(60) Provisional application No. 63/380,610, filed on Oct. 24, 2022.

(51) Int. Cl. A61K 8/92; A61K 8/34; A61K 8/37; A61K 8/67; A61K 8/73; A61K 8/9717; A61Q 1/00; A61Q 19/00

(52) U.S. Cl. CPC A61K 8/922; A61K 8/345; A61K 8/37; A61K 8/676; A61K 8/73; A61K 8/9717; A61Q 1/00; A61Q 19/00; A61K 2800/31; A61K 2800/87

(57) ABSTRACT

It is provided a skin care formulation comprising less than <1% in wt of water. Accordingly, the skin care formulation provided is packaged in a plastic-free container such as a cardboard container, and is made of ingredients from vegetable or mineral sources (natural), without water (<1%) and having a hardness value of up to 85 PU.

### CROSS-REFERENCE TO RELATED APPLICATION

The present application is claiming priority from U.S. Provisional Application No. 63/380,610 filed Sep. 15, 2022, the content of which is hereby incorporated by reference in its entirety.

### TECHNICAL FIELD

It is provided a skin care formulation comprising less than <1% in wt of water and packaged in a cardboard.

### BACKGROUND

There are many skin formulations available to consumers as it is a lucrative market. Many personal care compositions include ingredients which do not directly contribute to the actual purpose of the product other than providing particular rheological or other chemical and/or mechanical properties to the applied product.

For instance, several skin formulations include high levels of surfactants and alcohol, which can be caustic or irritating to skin. Further, the use of high amounts of water generally precludes the addition of other ingredients that can be beneficial to skin. Also, by using high amounts of water, packaging of such products is limited to plastic containers or glass jars.

Consumers are continually seeking personal care products providing improved performance benefits while minimizing undesirable environmental impacts.

It would be advantageous to be provided with formulations comprising only natural chemical additives.

### SUMMARY

It is provided herewith a skin care formulation comprising less than 1% in weight of water and a hardness value of up to 85 PU.

In an embodiment, the skin care formulation has a hardness value of between 20 to 85 PU.

In an embodiment, the formulation encompassed herein comprises at least one wax.

In another embodiment, the formulation encompassed herein comprises between 1 to 50% in wt of the at least one wax, more preferably comprising between 5 to 40% in wt of the at least one wax.

In an embodiment, the formulation encompassed herein comprises a sunflower wax, more preferably between 1 to 25% in wt of sunflower wax, or between 1 to 20% in wt of sunflower wax.

In an embodiment, the formulation encompassed herein comprises an oil, comprises between 1 to 50% in wt of oil, and more preferably between 5 to 50% in wt of oil.

In another embodiment, the oil grape seed oil. Alternatively, the oil is at least one of grape seed oil, evening primrose oil, argan oil, safflower oil, olive oil, moringa oil, avocado oil, sweet almond oil, jojoba oil, sunflower oil, castor oil, and a combination thereof.

In a further embodiment, the formulation encompassed herein comprises an emollient, preferably between 1 to 60% by wt of emollient, more preferably 5 to 55% by wt of emollient.

In a supplemental embodiment, the emollient is at least one of octyldodecanol, dibutyl lauroyl glutamide, dibutyl ethylhexanoyl glutamide, Celtiol® Ultimate, oleyl lactate, dicaprylil ether, caprylil glycol, magnolol, isopropyl palmitate, isostearyl isostearate, octyldodecyl myristate, caprylic, capric triglyceride, glyceryl caprylate, caprylhydroxamic acid, caprylate, and a combination thereof.

In a further embodiment, the formulation encompassed herein comprises an active molecule.

In another embodiment, the active molecule is at least one of zinc oxide, polyhydroxystearic acid, hydrolyzed soy protein, rice amino acids, L-proline, sodium benzoate, hydrolyzed adansonia digitata seed extract, glycerin, Nasturtium Officinale extract, glycogen, phytoglycogen, Laminaria Digitata extract, Marix limus (sea silt) extract, panthenol, Palmaria Palmata extract, plankton extract, Crithmum Maritumum extract, Ascophyllum Nodosum extract, Fucus Vesiculosus extract, Laminaria Saccharina extract, Ecklonia Cava extract, polyglyceryl-10 oleate, sorbitan palmitate, 3-o-ethyl ascorbic acid, Magnolol, Honokiol, Montmorillonite, peptide, arrow-root powder, and a combination thereof. Preferably, the formulation encompassed herein comprises between 0.15 to 15% by wt of active molecule, more preferably comprising between 0.55 to 5% by wt of active molecule.

In a further embodiment, the formulation encompassed herein further comprises a butter, such as e.g. at least one of Butyrospermum parkii (Shea) butter, olus oil (vegetable oil), and Theobroma cacao (Cocoa) seed butter.

In an embodiment, the formulation encompassed herein comprises at least 2%, more preferably between 7 to 22% by wt of butter.

In an embodiment, the formulation encompassed herein further comprises a surfactant.

In a particular embodiment, the surfactant is at least one of sucrose laurate, sucrose stearate, coco-glucoside, sodium cocoyl isethionate, polyglyceryl-6 stearate, polyglyceryl-6 behenate, and a combination thereof.

In an embodiment, the formulation encompassed herein comprises up to 14.5% by wt of surfactant.

In another embodiment, the formulation encompassed herein further comprises a humectant.

In an embodiment, the humectant is glycerin or propanediol.

In an embodiment, the formulation encompassed herein comprises up to 15% by wt of humectant.

It is also provided a skin care product comprising a packaging and the skin care formulation as defined herein.

In an embodiment, the packaging is a cardboard packaging. Preferably, the skin care formulation is packaged in the cardboard by hot filling.

In another embodiment, the product is a facial cream for day and/or night, a moisturizing eye cream, skin serum, a makeup remover, a face exfoliant, a face mask, a facial cleanser, a facial foundation, a facial tinted oil, a facial tanner, a facial dark circles concealer, a blush, a lip gloss, a facial illuminator, or a face oil product.

It is also provided a skin care formulation comprising less than 1 % in weight of water; a hardness value of up to 85 PU; between 1 to 25% in wt of sunflower wax; and between 1 to 50% in wt of oil.

### BRIEF DESCRIPTION OF THE DRAWINGS

Reference will now be made to the accompanying drawings.

FIG. 1 is a picture of two formats of a skin care formulation in accordance to an embodiment packaged in a cardboard.

### DETAILED DESCRIPTION

In accordance with the present description, there is provided a skin care formulation comprising less than <1% in wt of water. Accordingly, the skin care formulation provided is packaged in a plastic-free container such as a cardboard container, and is made of ingredients from vegetable or mineral sources (natural), without water (<1%).

The formulation described herein comprises less than <1% in wt of water and a hardness value of up to 85 PU. It is provided a kit comprising the formulation described herein and a cardboard packaging.

As described herein, the skin care formulation comprises at least one wax, preferably between 1 to 50% in wt of the at least one wax.

Sunflower wax is used for its properties of freeze/thaw resistance, for the harder structure. Sunflower wax provides interesting sensory texture and stability to the finished product in absence of water. On the contrary, candallila wax which is commonly used in the market did not result in products with satisfactory properties. It gave brittle, crumbly products that adhere to the cardboard packaging. In an embodiment, the formulation comprises between 1 to 25% in wt of sunflower wax.

It is also encompassed that the skin care formulation described herein further comprises an acticire or further wax. Acticire encompassed herewith can be formulated as comprising sunflower wax. Such further wax can be e.g. Jojoba Esters, Acacia Decurrens Flower wax, Euphorbia cerifera (Candelilla) wax, or Myrica cerifera (Bayberry) fruit wax. As provided herein, the formulation comprises between 5 to 36.5% by wt of a further wax.

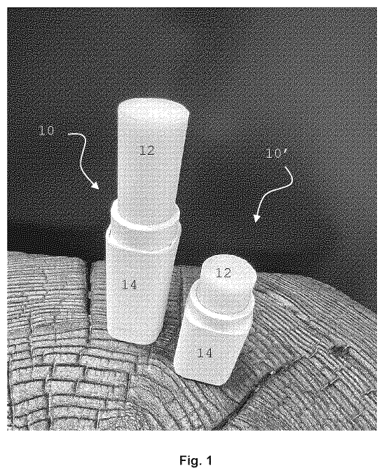

The formulation provided herein meet the following quality criteria which are density and hardness (measured with a penetrometer), and stability under various conditions (freeze/thaw, room temperature, 40 °C stability). The hardness value of the final product is up to 85 PU, preferably between 20 to 85 PU. In addition, the formulation provided herein allows packaging by hot filling in a cardboard because of its adhesion properties once solidified to the walls of the package. Minimum product absorption is noticed through packaging and there is no leakage under the container/cardboard packaging. As seen in FIG. 1, the skin care formulation 12 as encompassed herein can be packaged in different formats (10 and 10′) in a cardboard 12.

The formulation provided herein has the proper sensory effect on the skin when applied. Its surface appearance, homogeneity between the center and the sides of the product, as well as the top and the under portion of the formulation packaged, and smell are all as expected for a skin care formulation.

In an embodiment, the formulation comprises an oil, preferably grape seed oil. Grape seed oil is used because of its solidification point (fusion) of less than −18 °C, which allows to adjust freeze/thaw stability issues and maintain acceptable hardness despite time and temperature exposures. In a further embodiment, the oil can be selected from evening primrose oil, argan oil, safflower oil, olive oil, moringa oil, avocado oil, sweet almond oil, jojoba oil, sunflower oil, castor oil, and/or a combination thereof. As provided herein, the formulation comprises between 1 to 50% in wt of oil.

As provided herewith, the skin care formulation can comprise an emollient or gelling agent. In an embodiment, the emollient is selected from octyldodecanol, dibutyl lauroyl glutamide, dibutyl ethylhexanoyl glutamide, Celtiol® Ultimate, oleyl lactate, dicaprylil ether, caprylil glycol, magnolol, isopropyl palmitate, isostearyl isostearate, octyldodecyl myristate, caprylic, capric triglyceride, glyceryl caprylate, caprylhydroxamic acid, caprylate, and/or a combination thereof. As provided herein, the formulation comprises between 1 to 60% by wt of an emollient.

In a further embodiment, the skin care formulation can comprise a butter such as e.g. but not limited to, Butyrospermum parkii (Shea) butter, olus oil (vegetable oil), or Theobroma cacao (Cocoa) seed butter. As provided herein, the formulation comprises between 7 to 22% by wt of a butter. The acticire is used to disperse propandiol in oil. The advantage of propandiol in a product which does not contain water is to offer hydration (because propandiol is a humectant) and moreover, this ingredient increases the penetration of active ingredients into the skin making the product even more efficient.

As provided herewith, the skin care formulation comprises an active molecule. Such active molecule is selected from, but not limited to, zinc oxide, polyhydroxystearic acid, hydrolyzed soy protein, rice amino acids, L-proline, sodium benzoate, hydrolyzed adansonia digitata seed extract, glycerin, Nasturtium Officinale extract, glycogen, phytoglycogen, Laminaria Digitata extract, Marix limus (sea silt) extract, panthenol, Palmaria Palmata extract, plankton extract, Crithmum Maritumum extract, Ascophyllum Nodosum extract, Fucus Vesiculosus extract, Laminaria Saccharina extract, Ecklonia Cava extract, polyglyceryl-10 oleate, sorbitan palmitate, 3-o-ethyl ascorbic acid, Magnolol, Honokiol, Montmorillonite, peptide, or arrow-root powder. As provided herein, the formulation comprises between 0.15 to 15% by wt of an active ingredient.

In a further embodiment, the skin care formulation encompassed herein can comprise further a surfactant, such as but not limited to, sucrose laurate, sucrose stearate, coco-glucoside, sodium cocoyl isethionate, polyglyceryl-6 stearate and polyglyceryl-6 behenate. In an embodiment, the skin care formulation encompassed herein can comprises up to 14.5% by wt of a surfactant.

In a further embodiment, the skin care formulation encompassed herein can comprise a humectant such as e.g. but not limited to, glycerin or propanediol, and more particularly up to 15% by wt of a humectant.

On the contrary to known formulations, sodium stearate, which is known to give structure and make a solid liquid once cooled, when used in the formulation provide herewith, generated structure that did not offer the desired stability when packaged in a cardboard. Indeed, there was a lot of adhesion and the packaging soaked of the product. In addition, there was a phenomenon of syneresis. Similarly, sucrose laurate when mixed in the formulation described herein produced phase shifts and a texture that was too soft. Further known emulsifiers were also incorporated to improve the stability, without success.

Accordingly it is provided a skin care formulation which allows packaging in a cardboard through hot filling of a homogeneous suspension. In an embodiment, the skin care formulation is a facial cream for day and/or night, a moisturizing eye cream, skin serum, a makeup remover, a face exfoliant, a face mask, a facial cleanser or a face oil product.

### EXAMPLE I

### Preparation of a Facial Cream

In a main reservoir, an oil or a mixture of oil, such as safflower oil, sunflower oil, and/or grapeseed oil is/are added and heated up to for example 50-70 °C and mixed continuously at 200-300 RPM. Pelemol OL (oleyl lactate), and Cetiol OE (dicaprylil ether) are added subsequently to the main reservoir, where a constant agitation at 200-300 RPM and temperature of for e.g. 50-70 °C is maintained.

At this stage, Prodhysoft ABK (olus oil or vegetable oil) can also be added.

An acticire and Lipex shea (Butyrospermum parkii (Shea) butter) preheated are added to the main reservoir and agitation is slightly increased between 250-400 RPM to avoid foam formation.

In a separate container, Sucragel CF (commercial mixture of Glycerin, caprylic/capric triglyceride, water and sucrose laurate) is agitated in order to form a vortex. Sisterna SP70C (sucrose stearate) is added slowly into the vortex until homogenization is attain, resulting in a first premix.

The first premix is then added gradually to the main reservoir, at a temperature of about 50-70 °C. Agitation is again slightly increased between 300-500 RPM to avoid foam formation and insure homogenization of the mixtures. Once homogenization is attain, the temperature in the main reservoir is then increased to 80-85 °C and the sunflower wax is incorporated, still under agitation at 300-500 RPM, until homogenization. Kester wax BWR (Myrica Cerifera (Bayberry) fruit wax) and subsequently Purity 21C (Zea Mays corn starch—a structuring agent) are then incorporated, under a temperature of 80-90 °C and under agitation at 300-500 RPM, until homogenization. Afterwards, once the content of the main reservoir is homogenous, temperature is reduced to 50-70 °C

Once temperature is stabilized back at 50-70 °C, Cetiol Ultimate (undecane and tridecane—exfoliate agent), is incorporated, liquefying the mixture in the main reservoir. Cetiol Ultimate is incorporated under agitation at 300-400 RPM.

In a separate container, Canadian Dulse extract GLB-7189 (glycerin and Palmaria Palmate extract—active ingredients) is agitated to create a vortex. Panthenol is added gradually to the vortex until homogenization. Subsequently, in order, PhytoSpherix (phytoglycogen), CollaPlant Z NPNF (mixture of water, hydrolyzed soy protein/rice amino acids, L-proline, sodium benzoate and hydrolyzed Adansonia Digitata Seed Extract) and Nio-VCS vitamin C (Polyglyceryl-6 Laurate, Polyglyceryl-10 Oleate, Sorbitan Palmitate, 3-o-Ethyl Ascorbic Acid, Magnolol and Honokiol) are added to the vortex in order to produce a second premixture.

The second premixture is then added gradually to the main reservoir at a temperature of about 50-70 °C under an agitation of about 300-400 RPM.

Radigar CT-R (anti-oxidant containing caprylic, capric, triglycerides and Rosmarinus officinalis (Rosemary) Leaf Extract) and Spectrastat (emollient containing glyceryl caprylate, glycerin and caprylhydroxamic acid) are added at temperature of about 50-70 °C under an agitation of about 300-400 RPM producing the final homogenized cream product. The final cream product is controlled to ensure quality before packaging (white/cream color, uniform cream and a density of about 0.8-0.9).

Once the product is controlled and quality ensured, the product is transferred into a common filler apparatus. The temperature of the product is controlled and the product remains homogeneous during hot filling of the cardboard packages.

While the present disclosure has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

1: A skin care formulation comprising less than 1% in weight of water and a hardness value of up to 85 PU.

2: The skin care formulation of claim 1, comprising at least one wax.

3: The skin care formulation of claim 1, comprising between 1 to 50% in wt of the at least one wax.

4. (canceled)

5: The skin care formulation of claim 1, comprising a sunflower wax.

6: The skin care formulation of claim 5, comprising between 1 to 25% in wt of sunflower wax.

7. (canceled)

8: The skin care formulation of claim 1, comprising an oil.

9: The skin care formulation of claim 8, wherein said formulation comprises between 1 to 50% in wt of oil.

10. (canceled)

11: The skin care formulation of claim 8, wherein the oil is grape seed oil.

12. (canceled)

13: The skin care formulation of claim 1, further comprising an emollient.

14: The skin care formulation of claim 13, comprising between 1 to 60% by wt of emollient.

15. (canceled)

16: The skin care formulation of claim 13, wherein the emollient is at least one of octyldodecanol, dibutyl lauroyl glutamide, dibutyl ethylhexanoyl glutamide, Celtiol® Ultimate, oleyl lactate, dicaprylil ether, caprylil glycol, magnolol, isopropyl palmitate, isostearyl isostearate, octyldodecyl myristate, caprylic, capric triglyceride, glyceryl caprylate, caprylhydroxamic acid, caprylate, and a combination thereof.

17: The skin care formulation of claim 1, further comprising an active molecule.

18: The skin care formulation of claim 17, wherein said active molecule is at least one of zinc oxide, polyhydroxystearic acid, hydrolyzed soy protein, rice amino acids, L-proline, sodium benzoate, hydrolyzed adansonia digitata seed extract, glycerin, Nasturtium Officinale extract, glycogen, phytoglycogen, Laminaria Digitata extract, Marix limus (sea silt) extract, panthenol, Palmaria Palmata extract, plankton extract, Crithmum Maritumum extract, Ascophyllum Nodosum extract, Fucus Vesiculosus extract, Laminaria Saccharina extract, Ecklonia Cava extract, polyglyceryl-10 oleate, sorbitan palmitate, 3-o-ethyl ascorbic acid, Magnolol, Honokiol, Montmorillonite, peptide, arrow-root powder, and a combination thereof.

19-20. (canceled)

21: The skin care formulation of claim 1, further comprising a butter.

22: The skin care formulation of claim 21, wherein said butter is at least one of Butyrospermum parkii (Shea) butter, olus oil (vegetable oil), and Theobroma Cacao (Cocoa) seed butter.

23. (canceled)

24: The skin care formulation of claim 1, further comprising a surfactant.

25-26. (canceled)

27: The skin care formulation of claim 1, further comprising a humectant.

28-29. (canceled)

30: A skin care formulation comprising:
less than 1 % in weight of water;
a hardness value of up to 85 PU;
between 1 to 25% in wt of sunflower wax; and
between 1 to 50% in wt of oil.

31: A skin care product comprising a packaging and the skin care formulation of any one of claim 1.

32: The skin care product of claim 31, wherein the packaging is a cardboard packaging.

33-34. (canceled)
